(19) United States
(12) Patent Application Publication
(10) Pub. No.: US 2025/0157596 A1
(43) Pub. Date: May 15, 2025

(54) ARTIFICIAL INTELLIGENCE (AI)-BASED WORKSTATION MODULE FOR REPEAT DOSE TOXICITY ASSESSMENTS

(71) Applicant: Subhadra Dravida, Hyderabad (IN)

(72) Inventor: Subhadra Dravida, Hyderabad (IN)

(73) Assignee: TRANSCELL ONCOLOGICS PRIVATE LIMITED, Hyderabad, TG (IN)

(21) Appl. No.: 19/023,803

(22) Filed: Jan. 16, 2025

Related U.S. Application Data
(63) Continuation-in-part of application No. 18/235,895, filed on Aug. 21, 2023.
(63) Continuation-in-part of application No. 17/722,528, filed on Apr. 18, 2022.

(30) Foreign Application Priority Data
Feb. 15, 2022 (IN) 202241008032

(51) Int. Cl. G16C 20/70; G16C 20/30

(52) U.S. Cl. CPC G16C 20/70; G16C 20/30

(57) ABSTRACT

Artificial intelligence (AI)-based method for cytotoxicity and repeated dose toxicity assessment is disclosed. A human MicroPhysiological System (hMPS) simulates microenvironment of the human system to evaluate immune responses to a test substance. Phenotype data are generated from a micrograph by treating hMPS with increasing the test substance concentrations. Micrographs are analyzed and IC50 of the specific test substance is calculated and determined using AI system. The IC50 value from the graphs is compared with that from LDH assay on hMPS treated with increasing the test substance concentrations. A 5% to 20% IC50 dose is administered for 2-4 hours, followed by a two-day recovery, with test substance administration on days 1, 4, and 7. Further, cells are collected on day 10 for downstream analysis. Cytotoxicity percentage is evaluated using AI system and validated by RNA transcriptomics, generating heat maps of toxicity-related genes to provide insights into toxicity pathways.

### CROSS-REFERENCE TO RELATED APPLICATIONS

The present application is a continuation-in-part application of patent application Ser. No. 18/235,895 titled “DIGITAL ANIMAL FREE TESTING (DAFT) SYSTEM” filed on Aug. 21, 2023 in the United States Patent and Trademark Office, which is a continuation-in-part application of patent application Ser. No. 17/722,528 titled “A NON-ANIMAL HUMAN RELEVANT WORKSTATION SYSTEM AND METHOD FOR TESTING NEUROVIRULENCE AND NEUROTOXICITY IN VACCINES”, filed in the United States Patent and Trademark Office on Apr. 18, 2022, which further claims priority to Indian Patent Application No. 202241008032 filed on Feb. 15, 2022, the entire contents of which are hereby incorporated by reference for all purposes.

### TECHNICAL FIELD

The present invention generally related to a cytotoxicity assessment. More particularly, the present invention is related to an artificial intelligence (AI)-based workstation module for repeat dose toxicity assessments.

### BACKGROUND

Preclinical studies are mandated by Food and Drug Administration (FDA, U.S.A.) before initiating first-in-human (FIH) clinical trials. The preclinical study shows dose range finding and repeated dose toxicity crucial for investigational new drug (IND) application. Repeated dose toxicity is a toxicology study designed to evaluate the adverse effects of a substance when administered repeatedly to an animal over a specific period. The repeated dose toxicity study is essential for understanding the cumulative impact of chemicals, pharmaceuticals, or other compounds on biological systems. The duration of the study typically depends on the intended use of the substance, ranging from 28 days for sub-acute studies to 90 days for sub-chronic studies, and six months or longer for chronic studies. Animals used in repeated dose toxicity study includes rodents or non-rodents. The study helps to identify the dose-response relationship and determine the no-observed-adverse-effect level (NOAEL) which are crucial for establishing safe exposure levels of the substance.

However, the repeated dose toxicity using animal models could be time-consuming, ethically challenging, and often involves complex procedures. The traditional animal models often fail to accurately predict human immune responses that leads to ethical and financial concerns. The use of animals in research that does not lead to reliable human outcomes can be seen as a misuse of resources and may involve unnecessary suffering for the animals, particularly when alternative methods could provide more accurate results. Further, the differences in immune system structures between species could influence interactions with test substances that leads to altered toxicity values. Further, the differences between animal and human immune systems could lead to non-predictive or unreliable results.

Therefore, there is a need for a method for cytotoxicity and repeated dose toxicity assessment. The method needs to be convenient, time-efficient, and capable of addressing ethical challenges while eliminating the need for complex procedures. Additionally, the method needs to be able to provide a safer and more effective drug development and reduce the reliance on animal testing.

### SUMMARY

The present invention discloses an artificial intelligence (AI)-based method for cytotoxicity and repeated dose toxicity assessment. The method is utilized to determine IC50 and assess the effect of drug under a repeat dosing regimen. At one step, a human MicroPhysiological System (hMPS) is provided. The hMPS configured to simulate a microenvironment that mimics a human system. The hMPS incorporates bioassay processes to evaluate the function of the miniature living system using a test substance. The test substance or product is an antibody-drug conjugates (ADCs).

At another step, the human MicroPhysiological System (hMPS) is treated with increasing concentrations of the test substance and micrographs using inverted phase contrast microscope of the treated hMPS representing phenotype data are generated. At yet another step, the micrograph is analyzed by using an artificial intelligence (AI) based system comprising AI-based convolutional neural network (CNN) to quantify signals corresponding to transiently affected cells, apoptotic cells, necrotic cells. The AI system comprises a proprietary convolutional neural network (CNN) model configured to analyze individual cells and identify new phenotypes. The AI system is trained with one or more modules having plurality of training datasets with high-resolution 20× phase-contrast micrographs. The dataset comprises approximately 10000 images highlighting about 100,000 features categorized as healthy, dead, or affected cells includes the cells in shock, apoptotic, or necrotic states.

At yet another step, IC50 value is determined from a graph plotted with the concentration of the test substance on X-axis against the percentage of affected cells on Y-axis. The IC50 value represents the test substance concentration at which 50% of the cells are affected, as derived from the graph. The IC50 is a half-maximal inhibitory concentration.

At yet another step, the IC50 value derived from the graphs is compared with the IC50 value obtained from a lactate dehydrogenase (LDH) biochemical assay on the hMPS treated with an increasing the test substance concentration, for validation of IC50-derived cytotoxicity of the test substance using human MicroPhysiological Systems (hMPS). Further a 5% to 20% (for example, 10%) of the IC50 value derived for the test substance is utilized to evaluate the test substance-induced cytotoxicity in hMPS. At yet another step, 5% to 20% (for example, 10%) of the IC50 value is derived and treat the hMPS with this concentration for 2-4 hours (for example, 3 hours), followed by recovery on Day 1 by removing the drug product At yet another step, the method includes administering the test substance to the hMPS at 5% to 20% (for example, 10%) IC50 concentration for 2-4 hours (for example, 3 hours) and followed by recovery through removing the test substance added to the hMPS. The test substance administration is repeated on day 4, and day 7 and the cells are collected for downstream analysis on the day 10.

At yet another step, the cytotoxicity percentage is evaluated and calculated by processing hMPS micrographs using the AI system on the 10th day. At yet another step, the cytotoxicity percentage is validated by processing hMPS cell extracts to harvest ribonucleic acid (RNA) for transcriptomics and provides a transcriptomics data.

At yet another step, one or more supporting heat maps are generated using the transcriptomics data on known predictive genes involved in toxicity pathways. At yet another step, clinical surrogate report is generated for cytotoxicity with reduced time and ethical constraints compared to traditional animal studies. The predictive gene comprises one or more kidney-related genes, one or more liver-related genes, and one or more nervous system-related genes. The kidney-related genes includes kidney injury molecule-1 (KIM-1), neutrophil gelatinase-associated lipocalin (NGAL), cystatin, and clusterin. The liver-related genes includes alanine transaminase (ALT), aspartate aminotransferase (AST), bile salt export pump (BSEP), and CYP3A4. The nervous system-related genes includes glial fibrillary acidic protein (GFAP), neuron-specific enolase (NSE), S100B. Further, the chronic exposure nervous system-related genes induces cumulative changes in gene expression and epigenetic regulation including miRNA expression and methylation status.

### BRIEF DESCRIPTION OF THE DRAWINGS

FIG. 1 illustrates a flowchart of an artificial intelligence (AI)-based method for cytotoxicity and repeated dose toxicity assessment, according to an embodiment of the present invention.

FIG. 2 illustrates a flowchart outlining the steps to conduct cytotoxicity and repeated dose toxicity assessment, according to an embodiment of the present invention.

FIG. 3 illustrates a flowchart outlining the steps to conduct cytotoxicity assessment and steps to conduct repeated dose toxicity assessment using developmental and reproductive toxicity (DART) method, according to an embodiment of the present invention.

### DETAILED DESCRIPTION OF EXAMPLE EMBODIMENTS

The present invention discloses an artificial intelligence (AI)-based method for cytotoxicity and repeated dose toxicity assessment. The cytotoxicity assessment is the process to measure the ability of a test substance that damages or kill the cells. The repeated dose toxicity assessment is a process to evaluate the effect of repeated exposure of the test substance in a living organisms over a period. The repeated dose toxicity helps to determine the safe exposure levels and inform regulatory safety standards for chemicals, drugs, and other substances in the living organisms.

The method utilizes a digital animal free testing (DAFT) strategy for the repeated dose toxicity assessment. The DAFT strategy is an innovative new approach methodology (NAM) that aligns with the definitions established by a food and drug administration (FDA) and the Center for Drug Evaluation and Research (CDER) under the Modernization Act 2.0. The DAFT strategy serves as an ethical alternative for evaluating dose toxicity and assessing safety risks associated with the test substance.

Additionally, the DAFT strategy reduces variability and enhances the statistical significance of findings related to human health outcomes. The DAFT strategy adheres to the principles of a 3Rs that includes Replacement, Reduction, and Refinement in animal testing by producing human-relevant responses, which promotes ethical research practices. The DAFT approach provides more accurate and clinically relevant insights of immune responses by employing an advanced human MicroPhysiological System (hMPS) and an artificial intelligence (AI). The AI system approach addresses limitations of traditional methods including ethical dilemmas, high costs, and inconsistent results due to the inability of an animal models to replicate a human immune system.

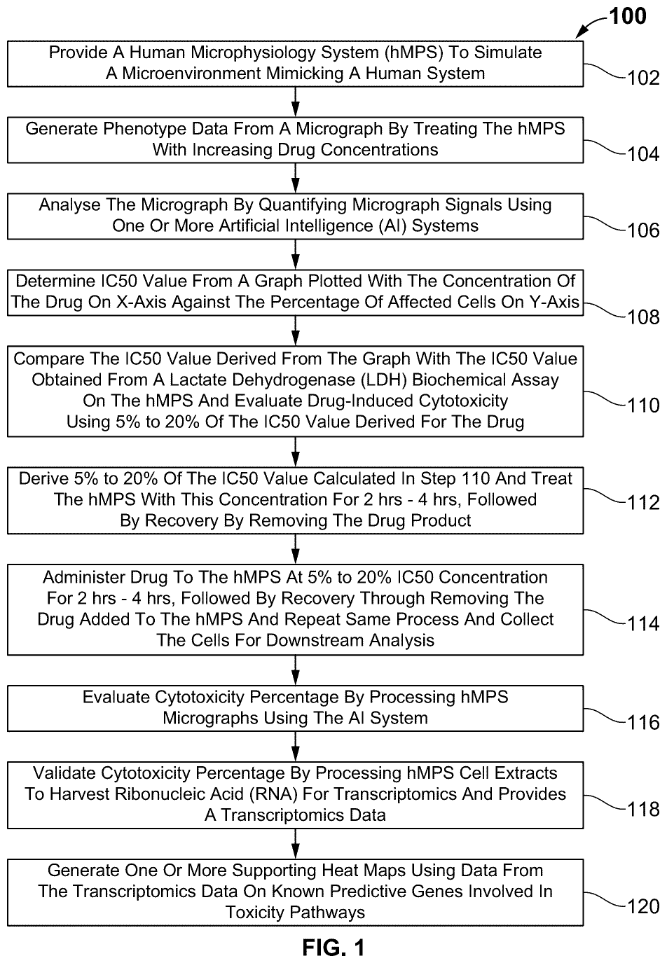

FIG. 1 illustrates a flowchart of an artificial intelligence (AI)-based method for cytotoxicity and repeated dose toxicity assessment, according to an embodiment of the present invention. At step 102, a human MicroPhysiological System (hMPS) is provided. The human MicroPhysiological System (hMPS) is configured to simulate a microenvironment that mimics a human system. The hMPS incorporates bioassay processes to evaluate the function of the miniature living system using a test substance. The test substance or product is an antibody-drug conjugates (ADCs). At step 104, the human MicroPhysiological System (hMPS) is treated with increasing concentrations of the test substance and generates one or more micrographs. The micrograph is a high-resolution image captured of the treated samples using microscopy techniques, for example phase-contrast microscopy. The micrographs representing phenotype data.

At step 106, the micrographs are analyzed using an artificial intelligence (AI) based systems. The artificial intelligence (AI) based system comprising AI-based convolutional neural network (CNN) to quantify signals corresponding to transiently affected cells, apoptotic cells, necrotic cells, and dead cells. The AI based system is an adaptable model integrates one or more modules for specific human MicroPhysiological System (hMPS) cells and bioassay protocols. Further, the AI-based convolutional neural network (CNN) model is configured to analyze individual cells and identify new phenotypes. The AI-based CNN model trained on high-resolution 20× phase-contrast micrographs to analyze individual cells and identify phenotypes, including healthy, dead, apoptotic, necrotic, and affected states. In one embodiment, the AI-based CNN model is trained using approximately 10,000 high-resolution phase-contrast micrographs with 100,000 features categorized as healthy, dead, or affected cells in shock, apoptotic, or necrotic states, enabling phenotype identification. The AI system predicts the percentage of affected cells in each image and enhances the understanding of cellular health and treatment effects. Further, validation was conducted using an independent dataset from wet lab experiments with curated drugs and vaccines, achieving a performance rate of 85%, with some predictions exceeding 100% based on positive control signals. Further, IC50 of the test substance is calculated using AI system by quantifying signals from the micrographs including affected cells, apoptotic cells, necrotic cells, and dead cells. The IC50 is a half-maximal inhibitory concentration.

At step 108, the IC50 value is determined from a graph plotted with the concentration of the test substance on X-axis against the percentage of affected cells on Y-axis. Further, the IC50 value represents the test substance concentration at which 50% of the cells are affected, as derived from the graph. At step 110, the IC50 value derived from the micrograph analysis is compared with a IC50 value obtained from a lactate dehydrogenase (LDH) biochemical assay on the hMPS treated with the increasing concentration of the test substance. Further, the IC50-derived cytotoxicity of the test substance is validated using human MicroPhysiological Systems (hMPS). At step 112, a 5% to 20% (for example, 10%) of the IC50 value calculated in step 110 is derived and the hMPS with 5% to 20% (for example, 10%) of the IC50 is treated for 2-4 hours (for example, 3 hours), followed by recovery on Day 1 by removing the drug product.

At step 114, the test substance is administered to the hMPS at a 5% to 20% (for example, 10%) IC50 concentration for 2-4 hours (for example, 3 hours) and followed by recovery through removing the test substance added to the hMPS. The test substance administration is repeated on 4^{th} day, and 7^{th} day. After the last recovery period, cells are collected on Day 10 (D10) for downstream analysis. At step 116, a cytotoxicity percentage is determined by processing hMPS micrographs using the AI system on the 10^{th} day. At step 118, the cytotoxicity percentage is validated by harvesting ribonucleic acid (RNA) to conduct transcriptomics and provides a transcriptomics data. At step 120, one or more supporting heat maps are generated using data from the transcriptomics data on known predictive genes relevant toxicity pathways. Further, clinical surrogate report is generated for cytotoxicity with reduced time and ethical constraints compared to traditional animal studies.

The predictive gene comprises one or more kidney-related genes, one or more liver-related genes, and one or more nervous system-related genes. The kidney-related genes includes kidney injury molecule-1 (KIM-1), neutrophil gelatinase-associated lipocalin (NGAL), cystatin, and clusterin. The liver-related genes includes alanine transaminase (ALT), aspartate aminotransferase (AST), bile salt export pump (BSEP), and CYP3A4. The nervous system-related genes includes glial fibrillary acidic protein (GFAP), neuron-specific enolase (NSE), S100B. The chronic exposure nervous system-related genes induces cumulative changes in gene expression and epigenetic regulation including miRNA expression and methylation status.

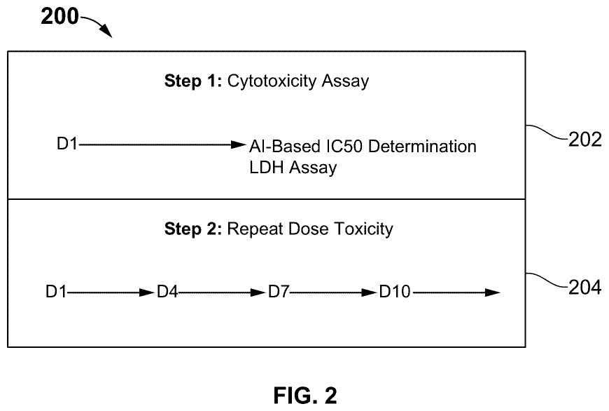

FIG. 2 illustrates a flowchart 200 outlining the steps to conduct cytotoxicity and repeated dose toxicity assessment, according to an embodiment of the present invention. At step 202, the cytotoxicity assessment determines the IC50 value using cytotoxicity assay. On day 1, the test substance is applied on the hMPS at increasing concentrations. Further, lactate dehydrogenase (LDH) assay and AI system is used to determine the IC50. In one embodiment, the cytotoxicity assessment is done based on the values of known information table. The known information table includes a monoclonal antibody (mAb) with a half-life of 14-21 days. The payload is the active ingredient in the test substance having an IC50 of approximately 2.9 nM and a half-life of 44 hours. The linker comprises a half-life of 15 hours. The antibody-drug conjugate (ADC) exhibits an IC50 of 2.5 nM.

At step 204, the repeat dose toxicity assessment evaluates the effect of the test substance when administered repeatedly over a specified period. The repeat dose toxicity assessment is conducted using 5% to 20% (for example, 10%) cytotoxicity of the test substance determined in step 202 is applied on the hMPS. The hMPS is treated in cycles of 3-hour test substance exposure followed by a 2-day recovery period is done on Day 1 (D1). The same treatment process is performed on Day 4 (D4), and Day 7 (D7). After the last recovery period, cells are collected on Day 10 (D10) for downstream analysis. The collected cells are used in downstream analysis for analyzing transcriptomics, phenomics, and cytotoxicity.

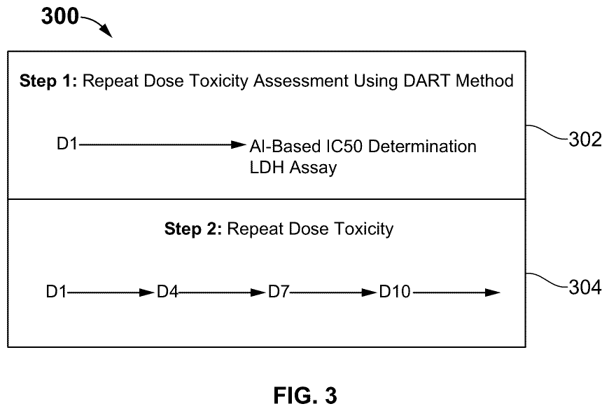

FIG. 3 illustrates a flowchart 300 outlining the steps to conduct cytotoxicity assessment and steps to conduct repeated dose toxicity assessment using developmental and reproductive toxicity (DART) method, according to an embodiment of the present invention. At step 302, the cytotoxicity assessment determines the IC50 value using cytotoxicity assay. On day 1, the test substance is applied on the hMPS at increasing concentrations. Further, the lactate dehydrogenase (LDH) assay and the AI system is used to determine the IC50. In one embodiment, the cytotoxicity assessment is done based on the values of known information table. The known information table includes a ref 1 with a half-life of 14-21 days, and a ref 2 having an IC50 of approximately 2.9 nM and a half-life of 44 hours. The table includes ref 3 of a half-life of 15 hours and a ref 4 exhibits an IC50of 2.5 nM.

At step 304, the repeat dose toxicity assessment evaluates the effect of the test substance when administered repeatedly over a specified period. The repeat dose toxicity assessment is conducted using 5% to 20% (for example, 10%) cytotoxicity of the test substance determined in step 302 is applied on the hMPS. The hMPS is treated in cycles of 3-hour test substance exposure followed by a 2-day recovery period is done on Day 1 (D1). The same treatment process is performed on Day 4 (D4), and Day 7 (D7). After the last recovery period, cells are collected on Day 10 (D10) for downstream analysis. The collected cells are used in downstream analysis for analyzing transcriptomics, phenomics, and cytotoxicity.

The downstream analysis of transcriptomics includes one or more kidney-related genes, one or more liver-related genes, and one or more nervous system-related genes. The kidney-related genes includes kidney injury molecule-1 (KIM-1), neutrophil gelatinase-associated lipocalin (NGAL), cystatin, and clusterin. The liver-related genes includes alanine transaminase (ALT), aspartate aminotransferase (AST), bile salt export pump (BSEP), and CYP3A4. The nervous system-related genes includes glial fibrillary acidic protein (GFAP), neuron-specific enolase (NSE), S100B. The chronic exposure nervous system-related genes induces cumulative changes in gene expression and epigenetic regulation including miRNA expression and methylation status. The downstream analysis of cytotoxicity includes lactate dehydrogenase (LDH) assay.

Advantageously, the method is convenient, time-efficient, and capable of addressing ethical challenges while eliminates the need for complex procedures. Further, the method comprising the AI system provide accuracy and reliable toxicity assessment. The AI-driven approach would enable researchers to predict and determine safer exposure levels of test substance with high precision and reduces the risks associated with human exposure. This AI-integrated system has the potential to transform toxicity testing by offering a scalable, ethical, and effective solution for both research and regulatory applications. The present method aligns with ethical standards and regulatory goals by replacing the need for animal testing.

We claim:

1. An artificial intelligence (AI)-based method for cytotoxicity and repeated dose toxicity assessment, comprising the steps of:
providing a human MicroPhysiological System (hMPS) configured to simulate a microenvironment, wherein the microenvironment mimics a human system;
treating a human MicroPhysiological System (hMPS) with increasing concentrations of the test substance and generating micrographs using inverted phase contrast microscope of the treated hMPS representing phenotype data;
analyzing the micrographs using an artificial intelligence (AI) based system comprising AI-based convolutional neural network (CNN) to quantify signals corresponding to transiently affected cells, apoptotic cells, necrotic cells, and dead cells, wherein the AI-based convolutional neural network (CNN) model is configured to analyze individual cells and identify new phenotypes;
deriving IC50 value from a graph plotted with a concentration of the test substance on X-axis against a percentage of affected cells on Y-axis, wherein the IC50 value represents the test substance concentration at which 50% of the cells are affected, as derived from the graph, and
comparing the IC50 value derived from the micrograph analysis with a IC50 value obtained from a lactate dehydrogenase (LDH) biochemical assay on the hMPS treated with the increasing concentration of the test substance, and validating IC50-derived cytotoxicity of the test substance using human MicroPhysiological Systems (hMPS).

2. The method of claim 1, further comprising steps of:
deriving 10% of the IC50 value calculated and treating the hMPS with 10% of the IC50 for 3 hours, followed by recovery on Day 1 by removing the drug product;
administering the test substance to the hMPS at 10% IC50 concentration for 3 hours and followed by a two days recovery through removing the test substance added to the hMPS, wherein the test substance administration is repeated on day 4, and day 7 and the cells for downstream analysis is collected on the day 10;
determining cytotoxicity percentage by processing hMPS micrographs using the AI system on the 10th day;
validating the cytotoxicity percentage by harvesting RNA from hMPS cell extracts to conduct transcriptomics and providing a transcriptomics data, and
generating one or more supporting heat maps using the transcriptomics data on known predictive genes relevant toxicity pathways.

3. The method of claim 1, further comprising step of: generating a clinical surrogate report for cytotoxicity with reduced time and ethical constraints compared to traditional animal studies.

4. The method of claim 1, wherein the AI-based CNN model is trained on high-resolution 20× phase-contrast micrographs to analyze individual cells and identify phenotypes, including healthy, dead, apoptotic, necrotic, and affected states.

5. The method of claim 1, wherein the AI-based CNN model is trained using approximately 10,000 high-resolution phase-contrast micrographs with 100,000 features categorized as healthy, dead, or affected cells in shock, apoptotic, or necrotic states, enabling phenotype identification.

6. The method of claim 2, wherein the predictive gene comprises one or more kidney-related genes, one or more liver-related genes, and one or more nervous system-related genes.

7. The method of claim 6, wherein the kidney-related genes include kidney injury molecule-1 (KIM-1), neutrophil gelatinase-associated lipocalin (NGAL), cystatin, and clusterin.

8. The method of claim 6, wherein liver-related genes include alanine transaminase (ALT), aspartate aminotransferase (AST), bile salt export pump (BSEP), and CYP3A4.

9. The method of claim 6, wherein the nervous system-related genes include glial fibrillary acidic protein (GFAP), neuron-specific enolase (NSE), S100B, wherein the chronic exposure nervous system-related genes induces cumulative changes in gene expression and epigenetic regulation including miRNA expression and methylation status.

10. The method of claim 1, wherein the test substance is an antibody-drug conjugates (ADCs).
